(19) United States
(12) Patent Application Publication
(10) Pub. No.: US 2008/0146650 A1
(43) Pub. Date: Jun. 19, 2008

(54) PHARMACEUTICAL COMPOSITION COMPRISING A-LIPOIC ACID FOR INFLAMMATORY DISEASES

(76) Inventors: Sung-Kwang Park, Seoul (KR); Won Kim, Seoul (KR); Six Lee, Seoul (KR); Sang-Ok Moon, Seoul (KR)

Correspondence Address: BIRCH STEWART KOLASCH & BIRCH, PO BOX 747, FALLS CHURCH, VA, 22040-0747, US

(21) Appl. No.: 12/034,434

(22) Filed: Feb. 20, 2008

Related U.S. Application Data
(62) Division of application No. 11/258,076, filed on Oct. 26, 2005.

(30) Foreign Application Priority Data
Sep. 29, 2005 (KR) 10-2005-91476

(51) Int. Cl. A61K 31/385; A61K 31/19

(52) U.S. Cl. 514/440; 514/557

(57) ABSTRACT

The present invention relates to a pharmaceutical composition containing α-lipoic acid (LA) as an active ingredient. α-lipoic acid is an inhibitor of fractalkine expression, and exhibits effects of alleviating inflammation due to endotoxemia by decreasing expression of fractalkine and attachment of endothelial cells to monocytes in endothelial cells of an LPS-induced endotoxemia model.

### BACKGROUND OF THE INVENTION

This application is a Divisional of co-pending U.S. application Ser. No. 11/258,076 filed Oct. 26, 2005, and for which priority is claimed under 35 U.S.C, §120; and this application claims priority of Application No. 10-2005-0091476 filed in the Republic of Korea on Sep. 29, 2005 under 35 U.S.C. §119; the entire contents of all are hereby incorporated by reference.

### FIELD OF THE INVENTION

The present invention relates to a pharmaceutical composition for treating endotoxemia. More specifically, the present invention relates to a therapeutic composition for treating endotoxemia, comprising an α-lipoic acid (LA), a compound which is effective to reduce endotoxemia due to LPS-induced fractalkine expression.

### DESCRIPTION OF THE RELATED ART

Sepsis is a clinical syndrome that represents the systemic response to the infection and characterized by systemic inflammation and widespread tissue injury. At the site of injury, the endothelium expresses various adhesion molecules which attract leukocytes (Cines D B et al, Blood 1998 91: 3527-3561). At the same time, inflammatory cells are activated and express a variety of adhesion molecules which cause their aggregation and margination to the vascular endothelium (Taub D D et al., Ther Immunol 1994 4: 229-246). When the inflammatory response is initiated, a wide variety of chemical mediators are released into circulation. These chemical mediators including TNF-α and IL-1β are associated with the continuation of the inflammatory response (Mantovani A et al., Immunol Today 1997 18: 231-240). Sepsisis caused mainly by an exaggerated systemic response to endotoxemia induced by gram-negative bacteria and their characteristic cell wall component, lipopolysaccharide (LPS) (Glauser M P et al., Lancet 1991 338: 732-736). In mice, challenge with high doses of LPS results in a syndrome resembling septic shock in humans (Gutierrez-Ramos J C et al., Immunol Today 1997 18: 329-334).

Gram-negative bacterial sepsis produces a spectrum of pathophysiological alterations, including cardiopulmonary, renal, hematologic, and metabolic dysfunction leading to vascular collapse (Levi M. et al., JAMA 1993 270: 975-979). Sepsis is associated with the induction of several cytokines which are proinflammatory and anti-inflammatory mediators. The excessive production of proinflammatory cytokines is thought to contribute significantly to lethality. Proinflammatory TNF-α and IL-1β act as initiators in the cascade of endogenous mediators that will direct the inflammatory and metabolic responses eventually leading to severe shock and organ failure.²⁷

Fractalkine (CX3CL1) is a structurally novel protein in which a soluble chemokine-like domain is fused to a mucin stalk that extends into the cytoplasm across the cell membrane.⁶ Fractalkine is expressed in the activated endothelial cells, and its expression is up regulated by TNF-α, IL-1β, and LPS (Harrison J K et al., J Leukoc Biol 1999 66: 937-944; Garcia G E et al., J Leukoc Biol 2000 67: 577-584). As a full-length transmembrane protein, fractalkine acts as an adhesion molecule and efficiently captures cells under physiological flow conditions (Haskell C A et al., J Biol Chem 2000 275: 34183-34189; Fang A M et al., J Exp Med 1998 188: 1413-1419).

However, cleavage of the fractalkine mucin stalk close to the junction of the transmembrane domain produces a soluble form of fractalkine that functions as a ligand of CX3CR1, a G-protein-coupled receptor (Imai T et al., Cell 1997 91: 521-530). In humans, CX3CR1 is expressed predominantly in monocytes, T cells, and NK cells. Thus, fractalkine and CX3CR1 have special roles in tethering and rolling, arrest, stable adhesion, and transendothelial migration of CX3CR1-expressing leukocytes at sites of fractalkine-expressing endothelium.

Vascular endothelial cells form a dynamically regulated barrier at the blood-tissue interface, and local factors generated by endothelial cell can be important pathogenic factors in inflammatory disorders such as sepsis. Fractalkine is a cell-surface anchored chemokine and has potent adhesive and chemotactic properties toward CX3CR1-positive cells. The important biological roles of fractalkine in endothelial inflammation and injury have been recently documented: firm adhesion of CX3CR1-positive cells cytotoxicity by the CX3CR1-expressing cytotoxic effector cells including NK cells, CD8⁺ T cells, and T cells; and enhanced effects of other chemokines on migration of CX3CR1-expressing cells into tissue (Yoneda O et al., J Immunol 2000 164: 4055-4062; Umehara H. et al., Arterioscler Thromb Vase Biol 2004 24: 34-40).

Activation of NF-κB could play a central role in inflammatory cytokine-induced fractalkine expression at the transcriptional level (Ahn S Y et al., Am J Pathol 2004 164: 1663-1672; Garcia G E et al., J Leukoc Biol 2000 67: 577-584). Our previous pharmacological assays revealed that TNF-α stimulated expression of fractalkine occurs mainly through activation of the NF-κB dependent pathway (Ahn S Y et al., Am J Pathol 2004 164: 1663-1672). It was also reported that SP-1 nuclear activator proteins are involved in vascular injury and inflammation (Silverman E S, Collins T. Am J Pathol 1999 154: 665-667).

Fractalkine expression is also markedly induced by inflammatory cytokines, such as IL-1β, and IFN-γ in primary cultured endothelial cells (Fraticelli P. et al., J Clin Invest 2001 107: 173-1181; Garcia G E et al., J Leukoc Biol 2000 67: 577-584). We previously reported that fractalkine is up-regulated after stimulation with TNF-α in HUVECs (Ahn S Y et al., Am J Pathol 2004 164: 1663-1672). Because fractalkine has important roles in inflammation, factors affecting its endothelial expression are important in regulating vascular inflammatory processes.

Endothelial cells are the primary targets of immunological attack in sepsis, and their injury can lead to vasculopathy and organ dysfunction (Yoneda O et al., J Immunol 2000 164: 4055-4062). Since inflammation is a universal pathogenesis in sepsis and LPS is a major pathogenic factor for the inflammatory response during gram-negative bacteremia, it is important to clarify the regulation of endothelial fractalkine expression in the prevention and treatment of the initial phase of endotoxemia.

α-lipoic acid (1,2-dithiolane-3-pentanoic acid) (LA), a disulphide derivative of octanoic acid, is a natural prosthetic group in α-keto acid dehydrogenase complexes present in the mitochondria. LA is known to act as an efficient antioxidant and metal-chelating agent (Suzuki Y J et al., Free Radic Res Commun 1991 15: 255-263; On P et al., Biochem Pharmacol 1995 50: 123-126). LA has been used to treat diabetic complications and polyneuropathies (Packer L et al., Nutrition 2001 17: 838-895; Ametov A S et al., Diabetes Care 2003 26: 770-776). LA also has been considered as a candidate of therapeutic agents in the treatment or prevent ion of pathologies that are associated with an imbalance of oxidoreductive status such as neurodegeneration (Gonzalez-Peres O et al., Neurosci Lett 2002 321: 100-104), ischemiareperfusion (Freisleben H J Toxicology 2000 148: 159-571), and hepatic disorders (Pari L, Murugavel P J Appl Toxicol 2004 24: 21-26). However, there is little data about the regulatory role of LA in fractalkine expression in endotoxemia.

### SUMMARY OF THE INVENTION

The present invention has been made in view of the above problems, and it is an object of the present invention to provide a pharmaceutical composition comprising α-lipoic acid which is useful for treatment of LPS-induced endotoxemia, as an active ingredient.

In accordance with an aspect of the present invention, the above and other objects can be accomplished by the provision of a pharmaceutical composition for inhibiting an inflammatory disease, comprising an α-lipoic acid, a pharmaceutically acceptable salt thereof or derivatives thereof, as an active ingredient to inhibit an inflammatory response in vascular endothelial cells.

In one embodiment, the present, invention provides an oral preparation of a pharmaceutical composition comprising α-lipoic acid (LA) and the oral preparation includes, but is not limited to, a tablet, a pill, a powder, a granule, a syrup, a solution, a suspension, an emulsion and a capsule.

In another embodiment, the present invention provides a parenteral preparation of a pharmaceutical composition comprising α-lipoic acid (LA) and the parenteral preparation includes, but is not limited to, an injectable preparation, a transrectal preparation and transdermal preparation.

In accordance with another aspect of the present invention, there is provided a method for treating an LPS-induced endotoxemic disease, comprising, administering to a host in need thereof, a therapeutically effective amount of the above compound.

### BRIEF DESCRIPTION OF THE DRAWINGS

The above and other objects, features and advantages of the present invention will be more clearly understood from the following detailed description taken in conjunction with the accompanying drawings, in which:

FIG. 1a graphically shows changes in serum TNF-α levels measured after intravenous injection of LPS into α-lipoic acid (LA)-pretreated rats and non-treated rats;

FIG. 1b graphically shows changes in serum. IL-1β levels measured after intravenous injection of LPS into α-lipoic acid (LA)-pretreated rats and non-treated rats;

FIG. 2a shows results of RNase protection assay (RPA) for expression of fractalkine due to stimulation of TNF-α in human umbilical vein endothelial cells (HUVECs), with respect to the passage of time;

FIG. 2b shows results of RNase protection assay (RPA) for expression of fractalkine due to stimulation of TNF-α in human umbilical vein endothelial cells (HUVECs), with respect to the passage of concentration;

FIG. 2c shows results of western blot analysis for expression of fractalkine due to stimulation of TNF-α in human umbilical vein endothelial cells (HUVECs), with respect to the passage of time;

FIG. 3a shows results of RNase protection assay (RPA) for expression of fractalkine due to stimulation of IL-1β in human umbilical vein endothelial cells (HUVECs), with respect to the passage of time;

FIG. 3b shows results of RNase protection assay (RPA) for expression of fractalkine due to stimulation of IL-1β in human umbilical vein endothelial cells (HUVECs), with respect to the passage of concentration;

FIG. 3c shows results of western blot analysis for expression of fractalkine due to stimulation of IL-1β in human umbilical vein endothelial cells (HUVECs), with respect to the passage of time;

FIG. 4a shows results of RNase protection assay (RPA) for expression of fractalkine due to stimulation of α-lipoic acid (LA) and TNF-α in human umbilical vein endothelial cells (HUVECs);

FIG. 4b shows results of RNase protection assay (RPA) for expression of fractalkine due to stimulation of α-lipoic acid (LA) and IL-1β in human umbilical vein endothelial cells (HUVECs);

FIG. 4c shows results of RNase protection assay (RPA) for expression of fractalkine due to stimulation of α-lipoic acid (LA), TNF-α and IL-1β in human umbilical vein endothelial cells (HUVECs);

FIG. 4d shows results of RNase protection assay (RPA) for expression of fractalkine due to stimulation of α-lipoic acid (LA), TNF-α and/or IL-1β in human umbilical vein endothelial cells (HUVECs);

FIG. 5a shows effects of LA on binding activation of NF-κB due to stimulation of TNF-α and IL-1β in HUVECs;

FIG. 5b shows effects of LA on binding activation of SP-1 due to stimulation of TNF-α and IL-1β in HUVECs;

FIG. 6a shows results of immunofluorescent staining on attachment of monocytes to HUVECs when HUVECs are treated with LA, TNF-α and fractalkine antibodies;

FIG. 6b shows quantitative results of immunofluorescent staining on attachment of monocytes to HUVECs when HUVECs are treated with LA, TNF-α, IL-1β and fractalkine antibodies;

FIG. 7a shows results of Immunohistochemical staining on LPS-induced monocyte attachment in vivo and

FIG. 7b shows quantitative results of Immunohistochemical staining on LPS-induced monocyte attachment in vivo.

### DETAILED DESCRIPTION OF THE INVENTION

Hereinafter, the present invention will be described in more detail.

The present invention encompasses a pharmaceutical composition for inhibiting an inflammatory disease, comprising α-lipoic acid, represented by Formula 1 or 2 as below, a pharmaceutically acceptable salt thereof or derivatives thereof, as an active ingredient to inhibit an inflammatory response in vascular endothelial cells.

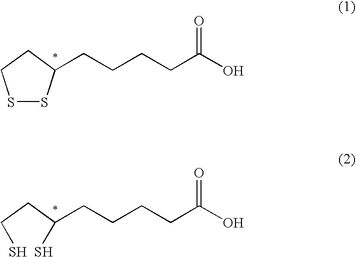

In connection with the compound in accordance with the present invention, the term “pharmaceutically acceptable salt” includes salts with pharmaceutically acceptable non-toxic bases or acids including inorganic or organic bases and inorganic or organic acids.

As used herein, the term “derivatives” includes a free hydroxyl, ethyl or methyl group.

The pharmaceutical composition in accordance with the present invention contains, as an active ingredient, an α-lipoic acid, or salts or derivatives thereof in an effective amount to prevent or treat an inflammatory response in vascular endothelial cells, wherein such compounds inhibit expression of fractalkine involved in the inflammatory response and decreases attachment of monocytes to endothelial cells, thereby alleviating inflammation.

The pharmaceutical composition in accordance with the present invention may be administered orally or parenterally. Although there is no particular limit to dosage of the pharmaceutical composition, it will be determined depending upon age of patients, sex or other conditions, severity of disease and dosage form. As examples of oral preparations, mention may be made of tablets, pills, powder, granules, syrup, solution, suspension, emulsion and capsules. As examples of parenteral preparations, mention may be made of injectable preparations, transrectal preparations and transdermal preparations, which may be administered intravenously, subcutaneously, intramuscularly, intraperiotoneally, etc. Preferably, the composition is administered orally.

In addition, the pharmaceutical composition in accordance with the present invention may be formulated into a desired dosage form by mixing the active ingredient with conventional pharmaceutically acceptable excipients, disintegrating agents, binding agents, lubricating agents, solubilizers, preservatives, stabilizers, buffers and coating agents.

For tablet formulation, a variety of carriers well-known in the art may be employed. Typical examples of carriers include, but are not limited to, excipients such as lactose, saccharose, sodium chloride, glucose, urea, starch, calcium carbonate, kaolin, crystalline cellulose and silicic acid binding agents such as water, ethanol, propanol, simple syrups, glucose solution, starch solution, gelatin solution, carboxy methyl cellulose, shellac, methylcellulose, potassium phosphate, polyvinyl pyrrolidone and sugar disintegrating agents such as dry starch, sodium alginate, agar powder, laminaran powder, sodium bicarbonate, calcium carbonate, polyoxyethylene sorbitan fatty acid esters, sodium lauryl sulfate, stearic acid monoglyceride, starches and lactose; disintegration aids such as saccharose, stearin, cacao butter and hydrogenated oils; absorption accelerators such as quaternary ammonium salts and sodium lauryl sulfate; humectants such as glycerin and starches; absorbents such as starches, lactose, kaolin, bentonite and colloidal silicic acid; and lubricating agents such as purified talc, stearate, boric acid powder and polyethylene glycol. Further, if necessary, the tablets may be coated (for example, sugar-coated tablets, gelatin-coated tablets, enteric-coated tablets, or film-coated tablets), and may be formed into double-layer tablets or multi-layer tablets.

For pill formulation, a variety of carriers well-known in the art may be employed. Useful carriers for use in pills include, but are not limited to, for example, excipients such as glucose, lactose, starches, cacao oil, butter, hydrogenated vegetable oils, kaolin and talc binding agents such as gum arable powder, tragacanth powder, gelatin and ethanol and disintegrating agents such as laminaran and agar.

When the composition of the invention is formulated into an injectable preparation, a sterile solution or suspension, which is isotonic with blood, is preferred. For formulation into solution, emulsion or suspension, any diluents, which are conventionally used in the art, may be employed. Examples of diluents that can be used in the present invention include water, ethyl alcohol, propylene glycol, ethoxylated isostearyl alcohol and propoxylated isostearyl alcohol and polyoxyethylene sorbitan fatty acid esters. In this connection, the injectable preparation may contain sodium chloride, glucose or glycerin in an amount sufficient to make an isotonic solution. Also, an ordinary solubilizer, buffer, smoothing agent or the like can be sufficiently added to the injectable preparation. Further, the preparation of the invention may contain coloring agents, preservatives, aromatic chemical agents, flavor, sweeteners, and other pharmaceutically acceptable additives, if necessary.

Where the composition is used for preventing or treating inflammatory diseases caused by septicemia, a dose of an active ingredient of the pharmaceutical composition in accordance with the present invention varies depending upon symptoms, age and weight of patients, the presence or absence of other conditions, and administration routes. For oral administration to an adult weighing 70 kg, the active ingredient of the pharmaceutical composition may be administered in a dose of 300 to 800 mg and preferably about 500 to 600 mg, singly or as a divided dose once or several times a day.

The pharmaceutical composition in accordance with the present invention may be prepared into various formulations, and can effectively prevent inflammation when administered to patients suffering from inflammatory diseases.

We found that the serum level of TNF-α after treatment with LPS (10 mg/kg intravenous by tail vein) increased as compared to that in control rats (0 pg/ml at 0, 1, 250±213 pg/ml at 1 hour, 1, 450±380 pg/ml at 2 hours, 975±121 pg/ml at 3 hours and 0 pg/ml at 4 hours after LPS). The serum level of IL-1β also increased compared to that in control rats (0 pg/ml at 0, 104±20 pg/ml at 1 hour, 232±45 pg/ml at 2 hours, 212±19 pg/ml at 3 hours and 127±12 pg/ml at 4 hours after LPS). These results suggest that the serum level of TNF-α and IL-1β increased during LPS-induced endotoxemia. Thus, we used TNF-α and IL-1β in this experiment.

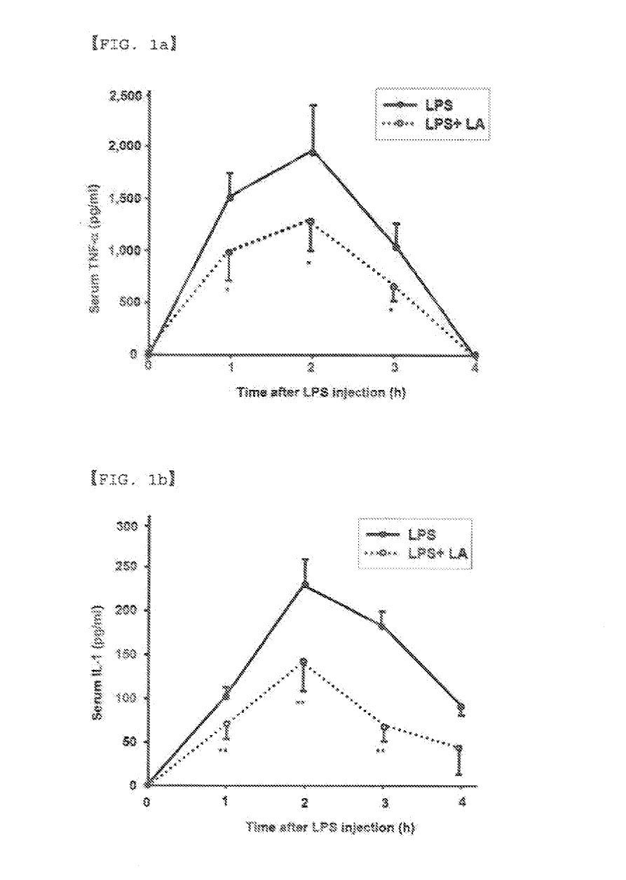

Further, the pharmaceutical composition of the present invention significantly inhibited TNF-α and IL-1β, expression of which was increased by administration of LPS in rats pretreated with LA (see FIGS. 1a and 1b). These results suggest that pretreatment with LA inhibits an increase in serum levels of TNF-α and IL-1β due to administration of LPS and thereby is correlated with anti-inflammatory effects.

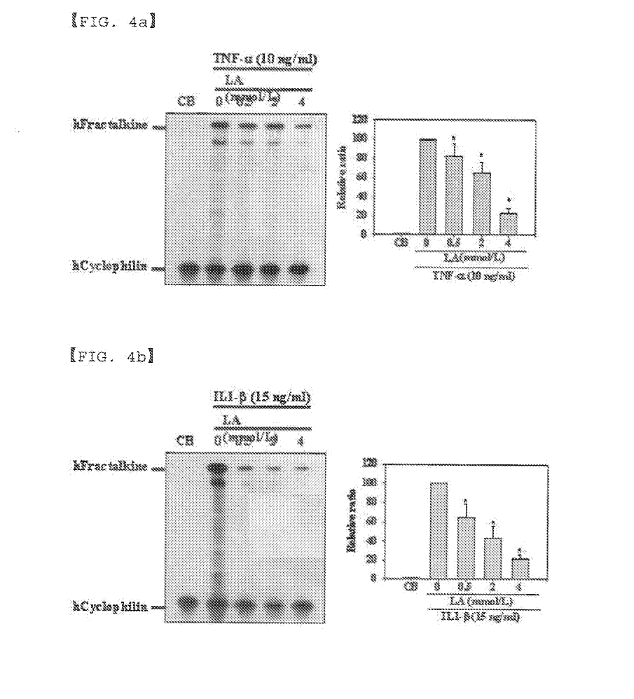

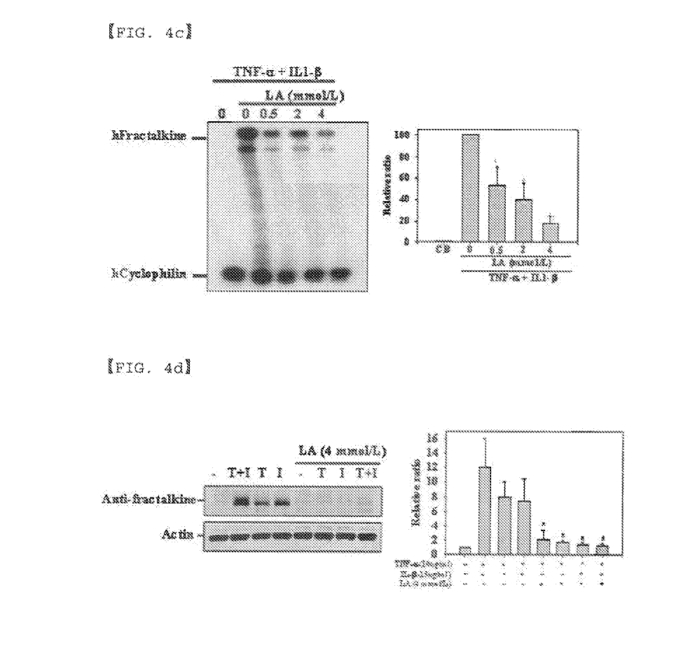

LA also decreased TNF-α- and/or IL-1β-induced expression of fractalkine protein (FIG. 4d). These data suggest that LA is an inhibitor of TNF-α, NF-κB and/or IL-1β-induced fractalkine expression in HUVECs.

Our EMSA indicated that LA suppressed not only NF-κB binding but also SP-1 binding of TNF-α- and/or IL-1β-stimulated endothelial proteins to the DNA. Therefore, it is possible that LA suppresses TNF-α- and/or IL-1β-induced fractalkine mRNA expression through suppression of NF-κB F-B and SP-1. Furthermore, our data demonstrated that incubation of confluent HUVECs with TNF-α or IL-1β caused an almost 5-fold or 4-fold increase in adhesion of monocytecells compared with adhesion of monocytes to unstimulated HUVECs. This increase in HUVEC adhesiveness was reduced by treatment with LA. Thus, LA has a regularoty role in fractalkine-mediated monocyte adhesiveness through suppression of NF-κB and SP-1.

Because challenge with high doses of LPS in rats results in a syndrome resembling human sepsis, a rat model of LPS-induced endotoxemia has been used in this study (Croner R S et al., Microvasc Res 2004 67: 182-191), Our immunohistochemical analyses in heart and intestine demonstrated that fractalkine was expressed slightly in arterial endothelial cells under normal conditions.

However, LPS increased fractalkine expression predominantly in arterial and capillary endothelial cells, while little or no induction of fractalkine expression was observed in venous endothelial cells. Furthermore, LPS increased fractalkine expression markedly in the endocardium of cardiac walls, the endocardial surfaces of cardiac valves, and the endothelium of intestinal villi.

Our data suggest that fractalkine expression intestine is endothelial cell-specific in endotoxemia. Considering the interaction between fractalkine-expressing endothelial cells and CX3CR1-expressing leukocytes in vivo, fractalkine must be involved in arterial inflammation rather than venous inflammation in endotoxemia. Pretreatment with LA dramatically suppressed LPS-induced fractalkine expression in arterial endothelial cells, endocardium, and endocardial surface of cardiac valves in heart. LA also decreased LPS-induced fractalkine expression in arterial endothelial cells and villous endothelium in small intestine. These data suggest that LA has a role in regulating fractalkine in arterial endothelial cells, endocardium, and the endocardial surface of cardiac valves in endotoxemia.

Our in vitro results have revealed that pretreatment with LA dramatically suppresses TNF-α- or/and IL-1β-induced fractalkine expression in endothelial cells through suppression of NF-κB and SP-1. Furthermore, LA decreases adhesiveness between cytokine-induced CX3CR1-positive leukocytes and endothelial cells through suppression of fractalkine expression. Our in vivo data also have demonstrated that LA decreased LPS-induced fractalkine expression in arterial endothelial cells, endocardium and villous endothelium. Therefore, LA warrants further evaluation as an anti-inflammatory drug in endotoxemia.

In the present invention, the inventors have investigated whether fractalkine is expressed in human umbilical vein endothelial cells (HUVECs), stimulated with TNF-α or IL-1β, or in arterial endothelial cells of an LPS-induced endotoxemia rat model. In addition, the inventors have investigated functions of lipoic acid in TNF-α or IL-1β-induced fractalkine expression in HUVECs, and in an LPS-induced endotoxemia model.

According to the present invention, α-lipoic acid inhibited NF-κB and SP-1 in HUVECs, thereby decreasing expression of fractalkine which is induced by TNF-α and IL-1β. Further, α-lipoic acid also inhibited attachment of endothelial cells to monocytes, which is induced by TNF-α or IL-1β.

According to the present invention, α-lipoic acid decreased expression of fractalkine in small intestine and myocardial arterial endothelial cells of an endotoxemia rat model. Such results suggest that α-lipoic acid is an effective agonist to reduce fractalkine-mediated inflammation in the endotoxemia model.

Now, construction and effects of the present invention will be described in more detail with reference to the following examples. These examples are provided only for illustrating the present invention and should not be construed as limiting the scope and spirit of the present invention.

### EXAMPLE 1

### Transient Elevation of Serum Levels of TNF-α and IL-1β After Intravenous Injection of LPS

Enzyme-linked immunosorbent assay (ELISA):

Blood samples (0.5 ml) were taken from rats at 0, 1, 2, 3, and 4 hours after the administration of LPS (10 mg/kg) or vehicle. Serum concentrations of TNF-α and IL-1β were determined by using ELISA kits (Endogen, Woburn, Mass.).

Serum concentrations of TNF-α and IL-1β were determined by using Enzyme-Linked Immunosorbent Assay (ELISA) Kits (Endogen, Woburn, Mass.). We found that the serum level of TNF-α after treatment with LPS (10 mg/kg intravenous by tail vein) increased as compared to that in control rats (0 pg/ml at 0, 1, 250±213 pg/ml at 1 hour, 1, 450±380 pg/ml at 2 hours, 975±121 pg/ml at 3 hours and 0 pg/ml at 4 hours after LPS). The serum level of IL-1β also increased compared to that in control rats (0 pg/ml at 0, 104±20 pg/ml at 1 hour, 232±45 pg/ml at 2 hours, 212±19 pg/ml at 3 hours and 12712 pg/ml at 4 hours after LPS). These results suggest that the serum level of TNF-α and IL-1β increased during LPS-induced endotoxemia. Thus, we used TNF-α- and IL-1β in this experiment.

Further, the pharmaceutical composition of the present invention significantly inhibited TNF-α and IL-1β, expression of which was increased by administration of LPS in rats pretreated with LA (see FIGS. 1a and 1b). These results suggest that pretreatment with LA inhibits an increase in serum levels of TNF-α and IL-1β due to administration of LPS and thereby is correlated with anti-inflammatory effects.

### EXAMPLE 2

### Induction of Fractalkine by TNF-α or IL-1β

1) Materials and Cell Culture

Recombinant human TNF-α was purchased from R&D Systems (Minneapolis, Minn.). Anti-fractalkine antibody was purchased from Torrey Pines BioLabs (Houston, Tex.). LPS was purchased from Sigma-Aldrich (St. Louis, Mo.). LA (Thioctacid 600®) was obtained from VIATRIS GmbH & Co. KG (Frankfurt, Germany). Calcein-AM was purchased from Molecular Probe (Eugene, Oreg.). Media, sera, and other biochemical reagents were purchased from Sigma-Aldrich, unless otherwise specified. HUVECs were prepared from human umbilical cords by collagenase digestion as previously described (Kim W et al., FASEB J. 2003 17: 1337-19395. Homogeneity of endothelial cells in cultures was confirmed by the presence of factor VIII using immunofluorescence method. HUVECs were maintained in M-199 medium supplemented with 20% (vol/vol) fetal bovine serum at 37° C. in a 5% CO₂atmosphere. The primary cultured cells used in this study were between 2 and 4 passages.

2) RNase Protection Assay (RPA)

A part of cDNA of human fractalkine (nucleotides 482-893, GenBank accession NM002996) was amplified by PCR and subcloned into pBluescript II KS+ (Stratagene, La Jolla, Calif.). After linearizing with EcoRI, ³²P-labeled antisense RNA probes were synthesized by in vitrotranscription using T7 polymerase (Ambion Maxiscript kit; Ambion, Austin, Tex.) and gel purified. RPA was performed on total RNAs using the Ambion RPA kit (Ambion, Austin, Tex.). An antisense RNA probe of human cyclophilin (nucleotides 135-239, GenBank accession X52856) was used as an internal control for RNA quantification.

3) Western Blot Analysis

Western blot analyses were performed as previously described (Aim S Y et al., Am J Pathol 2004 164: 1663-1672). Samples were mixed with sample buffer, boiled for 10 minutes, separated by SDS-PAGE electrotransfered to nitrocellulose membranes. The nitrocellulose membranes were blocked by incubation in blocking buffer, incubated with anti-fractalkine monoclonal antibody, washed, and incubated with horseradish peroxidase-conjugated secondary antibody. Signals were visualized using chemiluminescent reagents according to the manufacturer's protocol (Amarsham, Buckinghamshire, UK). The membranes were reblotted with anti-actin antibody to verify equal loading of protein in each lane.

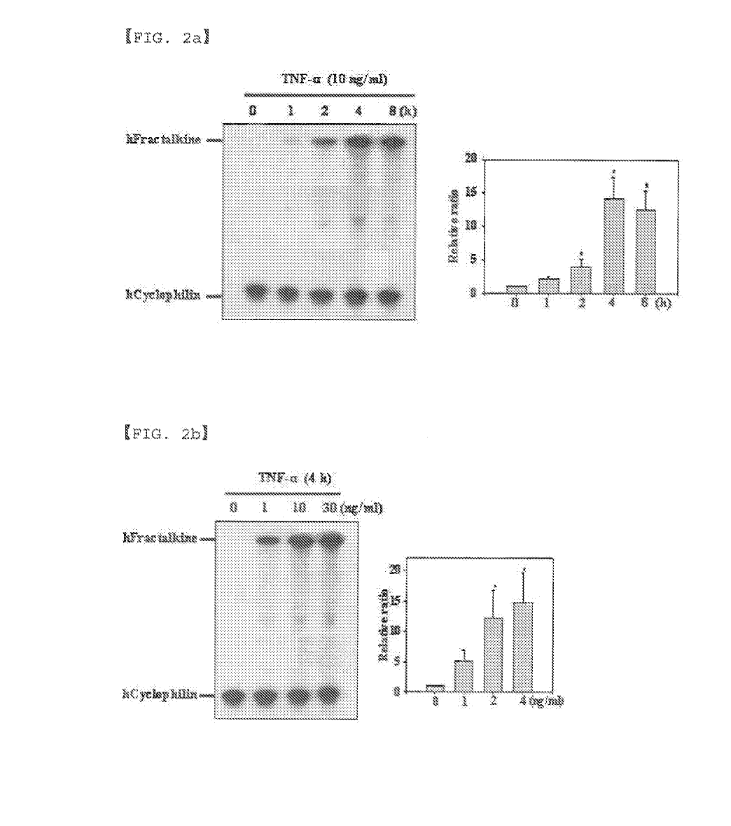

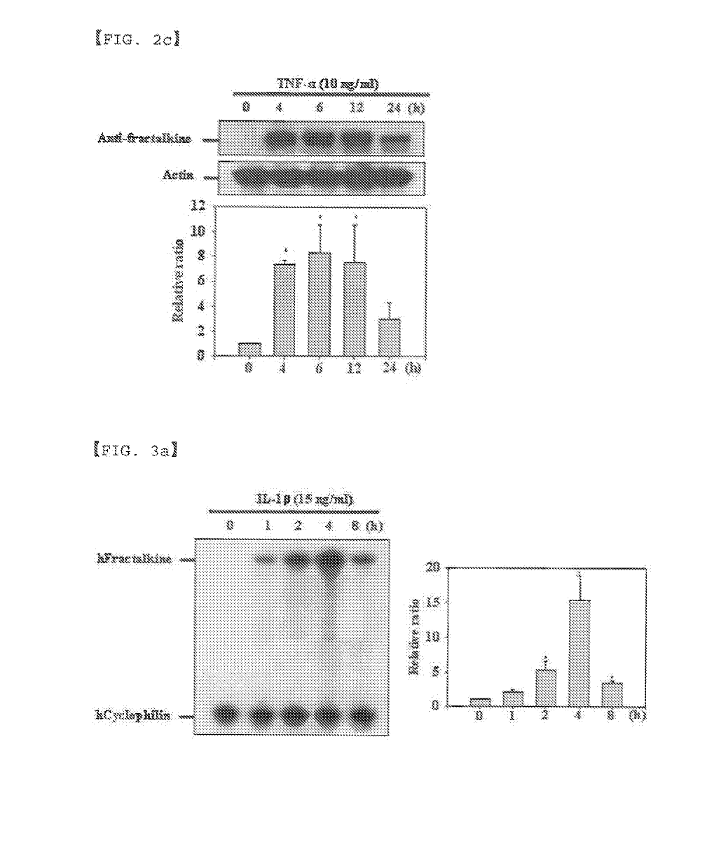

We firstly examined the effect of TNF-α and IL-1β on fractalkine expression in HUVECs. Addition of TNF-α (10 ng/ml) increased the expression of fractalkine mRNA in a time-dependent manner, and maximum expression of fractalkine was observed at 4 hours (FIG. 2a). The expression of fractalkine mRNA determined at 4 hour-incubation was increased in a dose-dependent manner of TNF-α (FIG. 2b). Consistent with the increased mRNA expression of fractalkine, fractalkine protein was also increased by treatment with TNF-α, and the level continued to he higher than control for up to 24 hours (FIG. 2c).

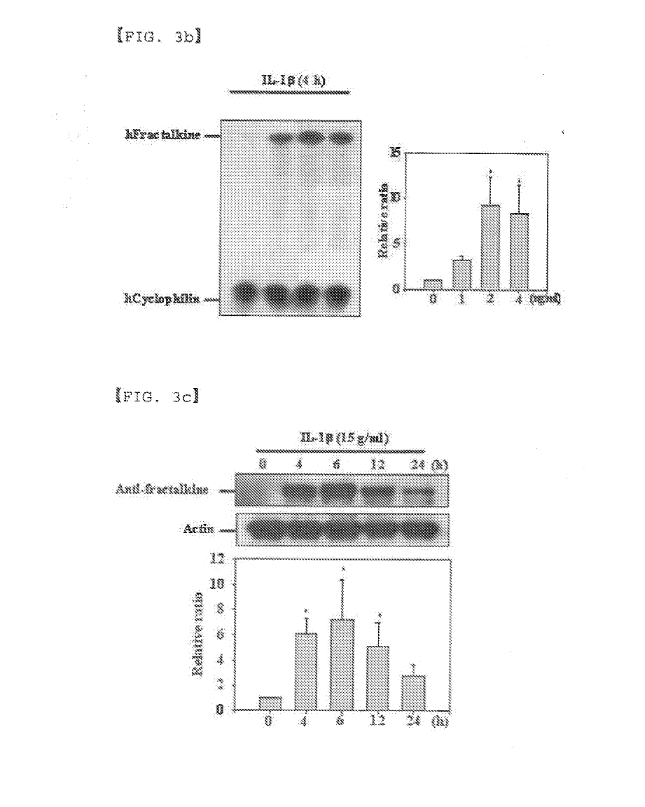

Treatment of HUVECs with IL-1β (15 ng/ml) gradually increased the expression of fractalkine mRNA up to 4 hours but a significant decrease in the fractalkine mRNA level was observed at 8 hours (FIG. 3a). The expression of fractalkine mRNA determined at 4 hour-incubation was increased in a dose-dependent manner of IL-1β (FIG. 3b). Maximum increase of fractalkine protein was observed at 4-6 hours and the level continued to be higher than control for up to 24 hours (FIG. 3c).

### EXAMPLE 3

### LA Suppressed TNF-α- and/or IL-1β-Induced Expression of Fractalkine mRNA and Protein

We examined the effect of LA on TNF-α- and/or IL-1β-induced fractalkine mRNA expression in HUVECs. LA (4 mmol/L) suppressed TNF-α (10 ng/ml)- or IL-1β (15 ng/ml)-induced expression of fractalkine mRNA in a dose-dependent manner (FIGS. 4a and 4b). LA suppressed approximately 70-80% of TNF-α or IL-1β-induced expression of fractalkine mRNA. Moreover, LA suppressed the expression of fractalkine mRNA induced by TNF-α (10 ng/ml) and IL-1β (15 ng/ml) together (FIG. 4c). LA also decreased TNF-α- and/or IL-1β-induced expression of fractalkine protein (FIG. 4a). These data suggest that LA is an inhibitor of TNF-α- and/or IL-IL-1β induced fractalkine expression in HUVECs.

### EXAMPLE 4

### Suppression of NF-κB and SP-1 Binding Activity in TNF-α- and/or IL-1β-Stimulated HUVECs Co-Treated with LA

EMSA (Electrophoretic Mobility Shift Assay):

EMSA for NF-κB proteins was performed as previously described (Kim I et al., J Biol Chem 2001 276: 7614-7620). Briefly, the cells were lysed in a hypotonic buffer (10 mmol/L HEPES, pH 7.9, 1.5 mmol/L MgCl₂, 10 mmol/L KCl, 0.5 mmol/L DTT, 0.5 mmol/L PMSF) containing 0.6% NP-40 and centrifuged at 4000 rpm for 15 min. The pellet was lysed in 15 l of a high salt buffer (20 mmol/L HEPES, pH 7.9, 420 mmol/L NaCl, 25% glycerol, 1.5 mmol/L MgCl₂, 0.2 mmol/L EDTA, 0.5 mmol/L PMSF, 0.5 mmol/L DTT) for 20 min on ice. Seventy five microliter of storage buffer (20 mmol/L HEPES, pH7.9, 100 mmol/L NaCl, 20% glycerol, 0.2 mmol/L EDTA, 0.5 mmol/L PMSF, 0.5 mmol/L DTT) was added, agitated for 10 sec by vortexing, and centrifused at 14,000 rpm for 20 min. Nuclear extracts (10 g) were incubated with approximately 20,000 cpm of ³²P-labeled NF-κB binding site oligomer 5′-AGTTGAGGGGACTTTCCCAGGC-3′ (SEQ ID NO: 1) (Santa Cruz Biotechnology, Santa Cruz, Calif.) for 30 man at 20 C. EMSA for SP-1 protein was performed with biotin-labeled SP-1 binding site oligomer 5′-GATCCGGTCCCCCACCATCCCCCGCCATTTCCA (SEQ ID NO: 2) and signals were detected by chemiluminescent imaging according to the manufacturer's protocol (EMSA Gel-Shift. Kit; Panomics, Redwood City, Calif.).

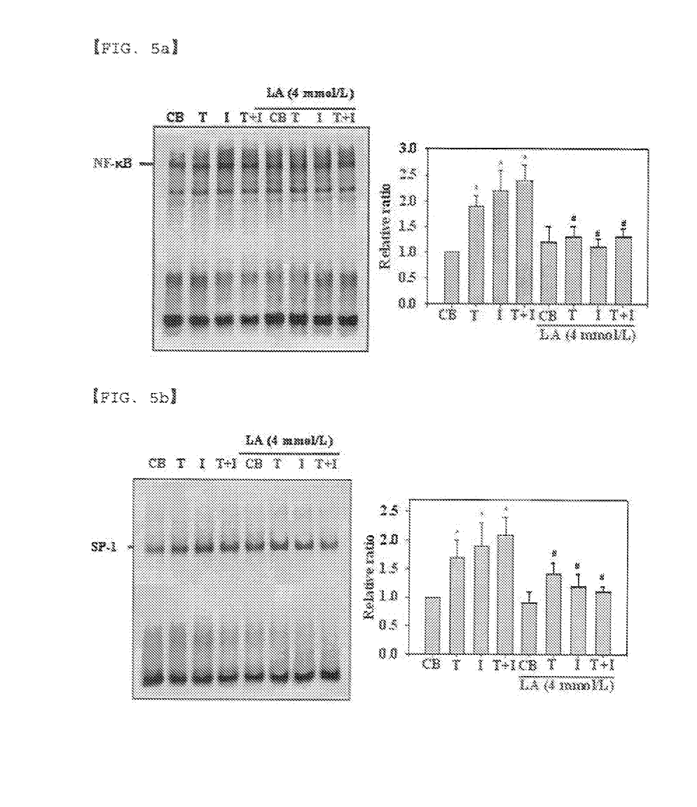

We previously reported that NF-κB is involved in TNF-α induced fractalkine expression in HUVECs (Ahn S Y et al., Am J Pathol 2004 164: 1663-1672). In this experiment, we examined whether LA inhibits NF-κB activity with the nuclear extracts of TNF-α (10 ng/ml)- and/or IL-1β (15 ng/ml)-stimulated HUVECs using electrophoretic mobility shift assay (EMSA). As shown in FIG. 5a, EMSA analyses revealed that NF-κB (p65/p50) binding activity was increased by the treatment with TNF-α and/or IL-1β band that LA (4 mmol/L) suppressed the TNF-α- and/or IL-1β-induced NF-κB (p65/p50) binding activity. LA alone had no effect on the basal NF-κB (p65/p50) binding activity. These data suggest that LA suppressed the TNF-α- and/or IL-1β-induced fractalkine expression through suppression of NF-κB activity in HUVECs.

Since TNF-α increase SP-1 binding activity in HUVECs and mithramycin, an inhibitor of SP-1, decreases TNF-α induced fractalkine expression in HUVECs, we examined whether LA can regulate SP-1 binding activity using to the nuclear extracts of TNF-α- and/or IL-1β-stimulated HUVECs (Ahn S Y et al., Am J Pathol 2004 164: 1653-1672; Shi J et al., J Cardiovasc Pharmacol 2004 44: 26-34). EMSA analyses revealed an increased SP-1 binding activity in HUVECs treated with TNF-α, IL-1β or TNF-α plus IL-1β. LA decreased the TNF-α- and/or IL-1β-induced SP-1 binding activity. LA alone had no effect on thebasal SP-1 binding activity (FIG. 5b). These data suggest that LA suppressed the TNF-α- and/or IL-1β-induced fractalkine expression through suppression of SP-1 activity in endothelial cells. Taken together, these data suggest that LA suppresses fractalkine expression by inhibiting NF-κB and SP-1 binding activities in HUVECs.

### EXAMPLE 5

### LA Suppressed TNF-α- or IL-1β-Induced Monocyte Adhesiveness to HUVECs

Monocyte isolation and adhesion assay:

Human peripheral blood monocytes were isolated from fresh blood of healthy volunteers by Ficoll-Paque gradient centrifugation. The study protocol and informed consent forms were approved by the Chonbuk. National University Hospital Review Board. Monocytes were isolated by negative selection using magnetic beads (Miltenyi Biotec, Bergisch Gladbach, Germany) (Ancuta P et al., J Exp Med 2003 197: 1701-1707). The purity of the monocyte fraction was 93-95% as determined by staining with anti-CD14, anti-CD33, anti-CD16b, and anti-CD56 mAbs and FACScan analysis (Becton Dickinson, Franklin Lakes, N.J.). Monocyte-endothelial adhesion was determined by fluorescent labeling of monocytes by a method described previously (Kim W. et al., Arterioscler Thromb Vase Biol 2003 23: 1377-1383). A number of monocytes adhered to HUVECs was expressed as percent calculated by the formula: % signal/total signal).

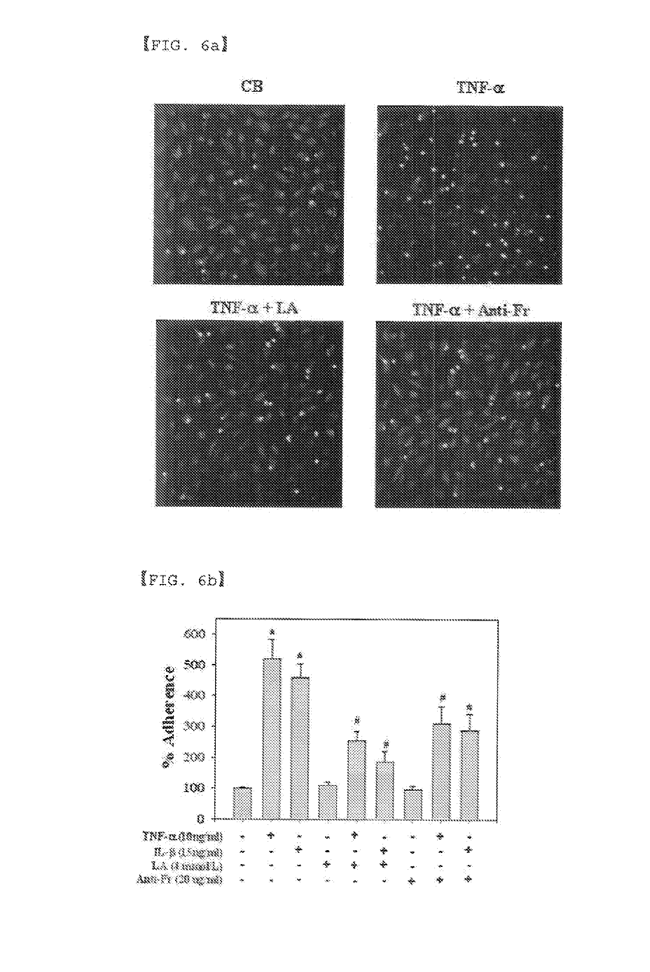

Expression of fractalkine in endothelial cells induces the adhesion of CX3CR1-positive cells such as monocytes (Imai T et al., Cell 1997 91: 521-530). We examined whether LA decreases monocyte adhesion to TNF-α- or IL-1β-stimulated HUVECs. A significantly increased adhesion of monocytes to HUVECs was observed in the presence of TNF-α or IL-1β. Stimulation of HUVECs with TNF-α (10 ng/ml) or IL-1β (15 ng/ml) for 6 hours induced a significant (5 or 4-fold each) increase in the adhesion of monocytes compared to treatment with control buffer. However, treatment of TNF-α-stimulated cells with LA led to a 63% decrease in monocyte adhesion and treatment of IL-1β-stimulated cells with LA led to a 76% decrease in monocyte adhesion (FIG. 6a, 6b). LA alone had no effects on HOVEC adhesiveness for monocytes. Moreover, the antibody against fractalkine decreased TNF-α- or IL-1β-stimulated monocytes adhesion (50% or 47% each). The fractalkine antibody alone had no effects on HUVEC adhesiveness for monocytes (FIG. 6a, 6b). These findings suggest that LA decreases monocyte adhesion to TNF-α- or IL-1β-stimulated HUVECs mainly through fractalkine expression.

### EXAMPLE 6

### LA Suppressed LPS-Induced Fractalkine Expression in Cardiac Endothelial Cells and Small Intestinal Endothelial Cells

1) Animal Experiments

Inbred male Sprague-Dawley rats (150-200 g) were obtained from Orient (Charles River Korea, Seoul, Korea) and were maintained on standard laboratory chow and water ad libitum. All animal studies were reviewed and approved by the Institutional Animal Care and Use Committee of Chonbuk National University Medical School. The rats (180-220 g) were divided into 3 groups; control (n=6), LPS (10 mg/kg) (n=6), and LPS (10 mg/kg) plus LA (10 mg/kg/day) (n=6). Control buffer and LPS were injected intravenously through the tail vein. LA was injected intraperitoneally once per day for three days prior to LPS administration. At 12 hours postinjection of vehicle or LPS, rats were anesthetized with ketamine (100 mg/kg) and xylazine (10 mg/kg), and subsequently sacrificed by cervical dislocation. Heart and jejunum were harvested for RNase protection assay, Western blot, and immunohistochemistry.

2) Immunohistochemical Analysis of Fractalkine Expression

After sacrifice, the hearts and jejunums were quickly excised, rinsed with PBS, and frozen in OCT in methyl-butane on dry ice. Frozen tissue blocks were sectioned at 10 m, and 8-12 sections of heart or jejunum from each rat were incubated with anti-fractalkine antibody at 4 C overnight. Signals were visualized with the Cell and Tissue Staining Kit (R&D Systems, Minneapolis, Minn.). The sections were counterstained with Meyer's hematoxylin and photographed using an Axioskope2 plus microscope (Carl Zeiss, Göttingen, Germany) equipped with color CCD camera (ProgResC14; Jenoptik, Jena, Germany) and monitor. Fractalkine expression was semi-quantitated by grading the degree of immunostaining (very strong=5, strong=4, moderate=3, weak=2, none=1). Three to five endothelial portions of each section were graded. Tissues were examined from several parts of the heart (artery, vein, endocardium and cardiac valves) and jejunum (artery, vein, and villous endothelium). Two independent, blinded investigators graded the expression by observation through a CCD camera. Inter-investigator variation was <5%.

We also examined the effect of LPS on fractalkine expression in rat heart using immunohistochemistry. Endogenous expression of fractalkine in normal adult rat was slightly observed in arterial endothelial cells, but almost no expression of fractalkine was observed in capillary endothelial cells, venous endothelial cells, endocardium, myocardium, pericardium, or cardiac valves. Intravenous injection of LPS (10 mg/kg) increased markedly fractalkine expression at 12 hours in arterial endothelial cells, endocardium, and endocardial surface of cardiac valves, but not in venous endothelial cells. Pretreatment with LA (10 mg/kg/day for 3 days) dramatically suppressed LPS-induced fractalkine expression in arterial endothelial cells, endocardium, and endocardial surface of cardiac valves.

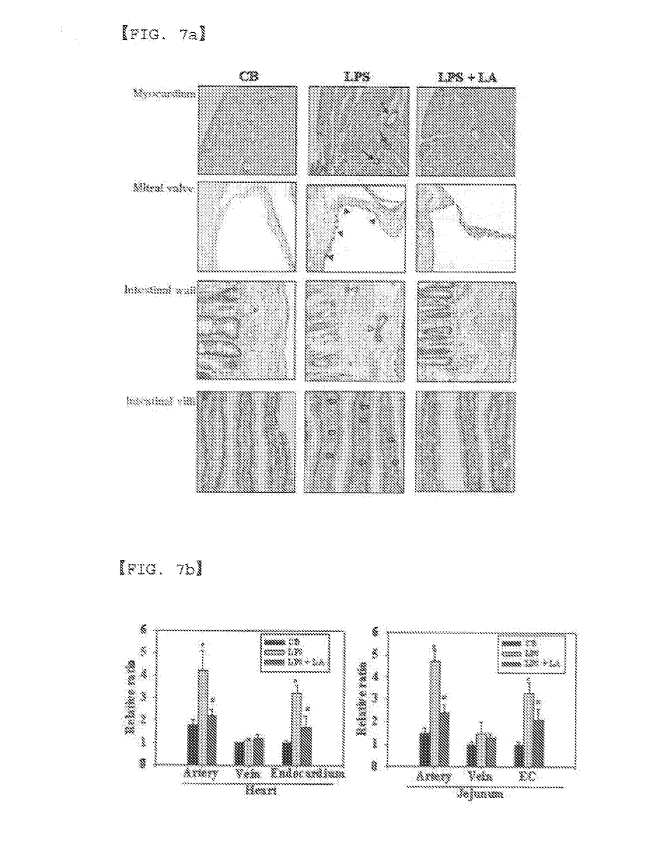

We further examined the effect of LPS on fractalkine expression in rat small Intestine using immunohistochemistry. We observed slight endogenous expression of fractalkine mainly in arterial endothelial cells, but only slight or almost no expression of fractalkine in villous endothelial, venous, and lymphatic endothelial cells, or epithelial cells (FIGS. 7a, 7b). Intravenous injection of LPS increased fractalkine expression markedly at 12 hours in arterial, arteriolar endothelial cells and villous endothelium, slightly in venous endothelial cells, but not in lymphatic endothelial cells or epithelial cells. These data suggest that LPS-induced fractalkine expression is endothelial cell specific in small intestine. Pretreatment with LA dramatically suppressed LPS-induced fractalkine expression in arterial endothelial cells and villous endothelium. These findings suggest that LA suppressed LPS-induced fractalkine expression in cardiac endothelial cells and small intestinal endothelial cells.

As apparent from the above description, pretreatment with α-lipoic acid inhibited expression of fractalkine in arterial endothelial cells of an LPS-induced endotoxemia model and thereby significantly inhibited attachment of monocytes to endothelial cells. These effects of α-lipoic acid are transmitted via an NF-κB signaling pathway and inhibit expression of fractalkine in arterial endothelial cells, endocardium, and the endocardial surface of heart valves. Therefore, it is expected that α-lipoic acid will be a useful material for development of a therapeutic agent effective to alleviate disease symptoms via inhibition of fractalkine-mediated inflammation in endotoxemia.

What is claimed is:

1. A method for treating LPS-induced endotoxemia, comprising, administering, to a host in need thereof, a therapeutically effective amount of a pharmaceutical composition comprising a α-lipoic acid (LA) of the following Formula 1, a dehydrolipoic acid of the following Formula 2 or a pharmaceutically acceptable salt thereof as an active ingredient.

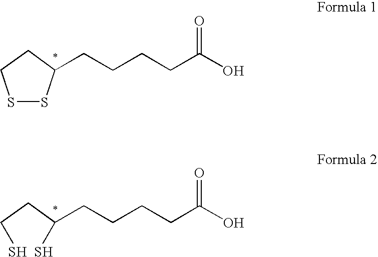

2. The method according to claim 1, wherein the active ingredient of the pharmaceutical composition is administered in a dose of 4.3 to 11.5 mg/kg.

3. The method according to claim 2, wherein the active ingredient of the pharmaceutical composition is administered in a dose of 7.2 to 8.6 mg/kg.

4. The method according to claim 1, wherein the active ingredient of the pharmaceutical composition is administered as a divided dose once or several times a day.

5. The method according to claim 1, wherein the active ingredient of the pharmaceutical composition is administered orally or parenterally.

6. The method according to claim 5, wherein the active ingredient of the pharmaceutical composition is administered intravenously, subcutaneously, intramuscularly or intraperiotoneally.


SEQUENCE LISTING (2 sequences)

SEQ ID NO: 1 - DNA, 22 bp, Artificial Sequence
  NF-kB binding site synthetic oligomer
agttgaggggactttcccaggc

SEQ ID NO: 2 - DNA, 33 bp, Artificial Sequence
  SP-1 binding site synthetic oligomer
gatccggtcccccaccatcccccgccatttcca